(19) United States
(12) Patent Application Publication
(10) Pub. No.: US 2022/0194718 A1
(43) Pub. Date: Jun. 23, 2022

(54) SYRINGE-REMOVAL PINCER WHEEL

(71) Applicant: Bausch + Ströbel Maschinenfabrik Ilshofen GmbH + Co. KG, Ilshofen (DE)

(72) Inventors: Andreas BÖHMIKE, Ilshofen (DE); Florian BÖRRET, Gaildorf (DE); Simon ACKERMANN, Frankenhardt (DE)

(73) Assignee: Bausch + Ströbel Maschinenfabrik Ilshofen GmbH + Co. KG, Ilshofen (DE)

(21) Appl. No.: 17/609,257

(22) PCT Filed: Apr. 30, 2020
(86) PCT No.: PCT/EP2020/061997

§ 371 Date: Nov. 5, 2021

(30) Foreign Application Priority Data
May 6, 2019 (DE) 10 2019 206 441.8

(51) Int. Cl. B65G 47/86; A61M 5/00

(52) U.S. Cl. CPC B65G 47/847; A61M 2209/04; A61M 5/002

(57) ABSTRACT

A device for receiving objects from a support on which the objects are arranged so as to lie side-by-side, in individual receptacles, in an axially parallel manner in rows. The device comprises a receiving zone. The device comprises a feed device for moving the support equipped with the objects through the receiving zone such that the objects arrive, in the sequence, at the receiving zone to be removed at the receiving zone, in the sequence, from the support; The device comprises a receiving wheel comprising grippers, the grippers being arranged around a periphery of the wheel and protruding outwards, wherein the receiving wheel is rotatable such that the grippers past the receiving zone one after another to receive, at the receiving zone, the objects, in sequence, from the support provided, wherein each gripper of the grippers is associated with a respective counterhold for securing a received object on the gripper.

The present invention relates to a device for receiving tubular or bottle-like objects, in particular syringe barrels, from a support on which the objects are arranged so as to lie side-by-side, in individual receptacles, in an axially parallel manner in rows.

It is known to provide syringe barrels in supports (trays) adjusted thereto, for further processing, the syringe barrels being arranged in the support so as to lie side-by-side, in individual receptacles, in an axially parallel manner in rows. The individual receptacles are generally designed such that they receive the syringe barrels in an interlocking and/or frictional manner.

For further processing of the syringe barrels, for example cleaning, sterilization, filling, said barrels must be released from the support and removed.

Hitherto it was entirely conventional to design devices of the type considered here such that they have simultaneously separated and removed all syringe barrels from their support, collisions and disruptive friction effects having sometimes occurred between the syringe barrels in practice. An example of a device of this kind comprising simultaneous removal of all syringe barrels from the tray is described for example in DE 19604100 C2.

The object of the present invention is that of demonstrating a new way of how tubular or bottle-like objects, for example syringe barrels, can be removed individually and gently from a tray.

In order to achieve this object, a device according to claim 1 is proposed, specifically a device for receiving tubular or bottle-like objects, in particular syringe barrels, from a support on which the objects are arranged so as to lie side-by-side, in individual receptacles, in an axially parallel manner in rows, and so as to be held in an interlocking and/or frictional manner. The device comprises a receiving zone in which the objects are to be removed from the support in sequence, a feed device for moving the support, equipped with the objects, through the receiving zone, such that the objects arrive at the receiving zone in sequence in order to there be removed from the support in sequence, and a receiving wheel comprising grippers that are arranged all around the periphery of said wheel and protrude outwards, the receiving wheel being arranged so as to be rotatable such that the grippers pass the receiving zone one after the other, in order to there receive objects in sequence from the support provided in each case, each gripper being associated with a respective counterhold for securing a received object on the gripper.

Of course, in this case, the term “receiving” can also be understood within the context of insertion. Furthermore, in addition to tubular and bottle-like objects, it would in principle be possible for general pharmaceutical packaging to be received using the device according to the invention.

Preferably, each gripper, together with the associated counterhold thereof, constitutes a pincer for receiving a relevant object from the support, in the receiving zone. Said pincers always receive just one object in the receiving zone, in the tangential region of the receiving wheel, such that subsequent objects are not influenced.

Each gripper preferably comprises a distal hook-like portion for engaging below an object in the support during passage through the receiving zone. In this way, reliable and defined reception of the object from the relevant individual receptacle of the support is possible, without applying critical friction to the object.

According to a particularly preferred embodiment of the invention, the grippers and the counterhold associated therewith in each case are movable relative to one another, in a controlled manner, so as to perform an opening and closing movement on the receiving wheel, by means of a control assembly, in order to open the pincer thus formed for receiving an object in the receiving zone and in order to close said pincer for securing the received object.

The control assembly preferably comprises a cam disk controller of the movement of the relevant gripper relative to the counterhold associated therewith.

Furthermore, it is preferably possible for the device to comprise means for controlled and mutually coordinated movement of the support through the receiving zone and the rotational movement of the receiving wheel during reception of the objects from the support, the movement of the support and the rotational movement of the receiving wheel taking place continuously. Alternatively, however, it would readily also be possible for clocked operation of the device according to the invention to be provided, in which the feed movement of the support and the rotational movement of the receiving wheel would have to be correspondingly mutually coordinated.

Embodiments of the invention will be explained in greater detail in the following, with reference to the drawings.

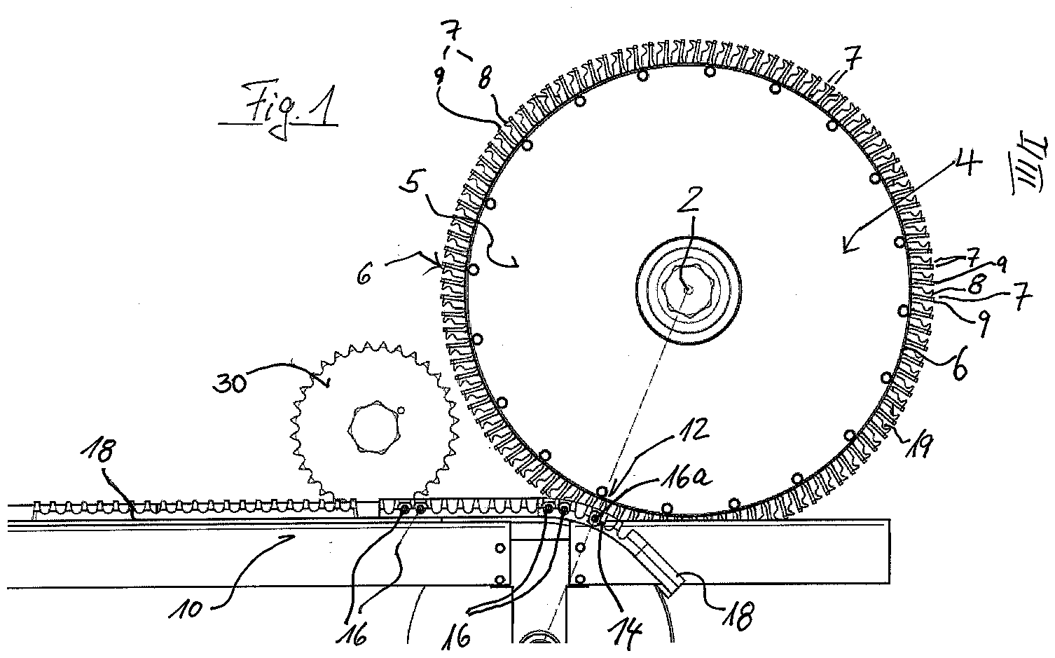

FIG. 1 is a partially schematic side view of an embodiment of the device according to the invention, a housing part normally laterally covering the transition zone being omitted.

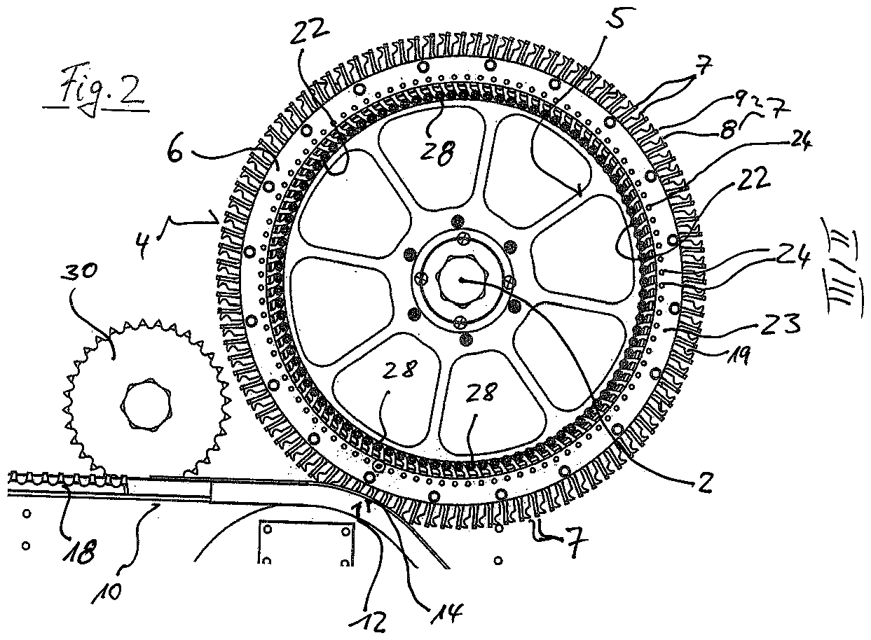

FIG. 2 is a corresponding side view of the embodiment of FIG. 1, a faceplate normally laterally covering the central fixed hub region of the receiving wheel being omitted.

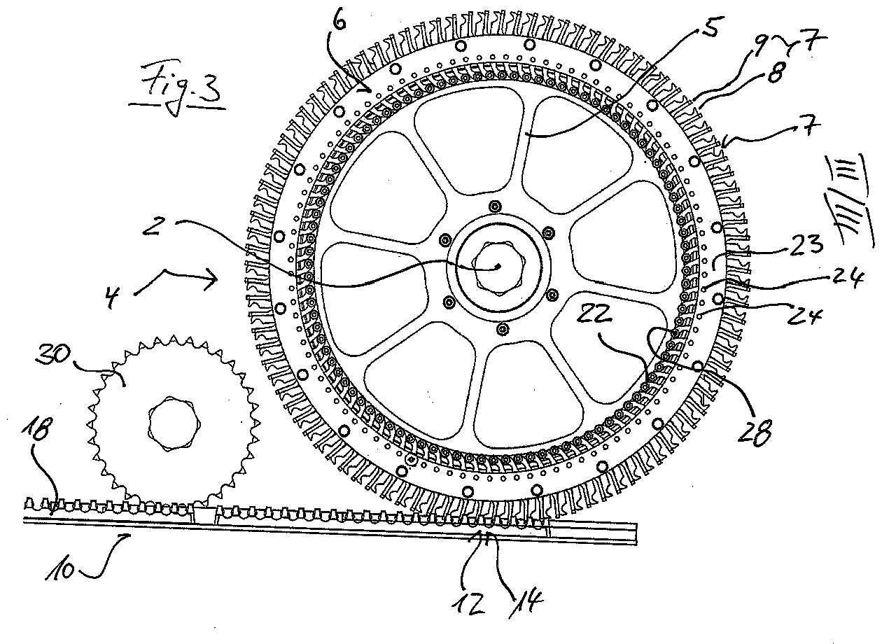

FIG. 3 is a partially schematic side view of a second embodiment of a device according to the invention, the central faceplate of the receiving wheel being omitted.

FIG. 1 and FIG. 2 show a receiving wheel comprising a central, fixed hub part 5 and a wheel rim 6 that is rotatable about the hub part 5, in the anticlockwise direction, about a horizontal axis of rotation 2, comprising pairs 7 of grippers 8 and the counterholds 9 associated with the grippers in each case, which pairs protrude outwards and are arranged all around the periphery thereof.

In the drawing according to FIG. 1 and FIG. 2, a support feeding device 10 first extends horizontally, from left to right, towards a receiving zone 12, and comprises a curvature having a rightwards and downwards course in the region of the receiving zone 12.

Reference sign 14 refers to an almost tangential region at which the course of the support feeding device 10 comes closest to the receiving wheel 4, at the periphery thereof. In this case, this is the center of the receiving zone 12, in which, in the example, syringe barrels 16 are received individually and in sequence by the receiving wheel 4, as objects, from a relevant support 18 fed in along the support feeding device 10. Just some syringe barrels 16 are shown in FIG. 1, even if, in practice, the supports 18 conveyed along the support feeding device 10 to the receiving wheel 4 are fully equipped with a complete group of syringe barrels 16.

The syringe barrel denoted 16a in FIG. 1 is currently located in the center of the receiving zone 12 and is removed from the support 18 by the receiving wheel 4 for further movement. Each pair 7 of the gripper 8 and counterhold 9 forms a pincer 7 for grasping a relevant syringe barrel 16.

For the purpose of receiving the relevant syringe barrel 16 by a pincer 7 consisting of a gripper 8 and counterhold 9, in the receiving zone 12 the grippers 8 and the associated counterhold 9 are movable relative to one another, in a controlled manner, so as to perform an opening and closing movement, by means of a control assembly 22 (FIG. 2), in order to open the pincer 7 in the receiving zone 12 and in order to close said pincer for securing the received syringe barrel 16. A device for receiving the relevant syringe barrels 16 from the receiving wheel 4 for the purpose of conveying the syringe barrel 16 further, to a subsequent processing station, is not shown in the drawings.

The control assembly 22 is designed as a cam disk controller, which can be seen in part in FIG. 2 for the first embodiment of the invention, and in FIG. 3 for the second embodiment of the invention.

In the case of each pincer 7 consisting of the gripper 8 and counterhold 9, the grippers 8 provided with a hook-like outer portion 19, and the counterholds 9, are mounted on the peripheral ring 23 of the receiving wheel 4 so as to be pivotable about a respective common swivel pin 24, and are spring-preloaded, by means of a relevant torsional spring (not shown), into a common basic position (in particular pincer closed position).

The grippers 8 comprise respective cam followers 28 on the ends thereof closest to the receiving wheel center 2, by means of which followers they are supported on the periphery of the central hub-like round element 5, under the pressure of the torsional spring. The periphery of the central round element 5 forms a surrounding cam disk which is shaped such that, upon entry into the receiving zone 12, the grippers 8 are applied to the respective cam followers 28 thereof such that they transition into an open position for receiving a syringe barrel 16 in the receiving zone 12, in order to receive a relevant syringe barrel 16. In the event of further movement of the receiving wheel 4, the relevant pincers 7 again arrive at the closed position thereof, in order to secure a syringe barrel 16, received in each case, on the receiving wheel 4.

In FIG. 1-3, a relevant pre-centering wheel is denoted 30, which wheel comes into meshed engagement, in FIG. 1-3, with supports 18 supplied from the left, and is rotatable in a controlled manner, such that it pushes forward the support 18, fed-in in each case, in the desired operational phase position, to the pincers 7 moved in the receiving zone 12, such that the pincers 7 find a relevant syringe barrel 16 in the receiving zone 12, for reception, and the supports 18 are fed-in behind one another at desired spacings. In the first embodiment according to FIGS. 1 and 2, the supports 18 are guided, in the region of the receiving zone 12, such that they undergo bending. For this purpose, they are formed so as to be elastically deformable in the corresponding manner. In the second embodiment according to FIG. 3, the support 18 passes through the receiving zone 12 in a straight line. Otherwise, however, the two embodiments substantially correspond to one another, such that the above description applies, in this respect, to both embodiments.

1. A device for receiving objects from a support on which the objects are arranged so as to lie side-by-side, in individual receptacles, in an axially parallel manner in rows, the device comprising:
a receiving zone in which the objects are to be removed from the support in sequence;
a feed device for moving the support equipped with the objects through the receiving zone such that the objects arrive, sequentially, at the receiving zone to be removed at the receiving zone, sequentially, from the support;
a receiving wheel comprising grippers, the grippers being arranged around a periphery of the wheel and protruding outwards, wherein the receiving wheel is rotatable such that the grippers pass the receiving zone one after another to receive, at the receiving zone, the objects, sequentially, from the support provided, wherein each gripper of the grippers is associated with a respective counterhold for securing a received object on the gripper.

2. The device of claim 1, wherein each gripper comprises a distal hook-like outside portion for engaging below an object in the support during a passage of the gripper through the receiving zone.

3. The device of claim 1, wherein each gripper together with the respective counterhold, forms a pincer-like structure for receiving an object from the support in the receiving zone.

4. The device of claim 3, wherein the grippers and the counterholds associated with the grippers are movable relative to one another, in a controlled manner, so as to perform an opening and closing movement on the receiving wheel using a control assembly, (A) to open the pincer-like structure for receiving an object in the receiving zone and (B) to close said structure for securing the received object.

5. The device of claim 4, wherein the control assembly comprises a cam disk controller to control a movement of a relevant gripper relative to the counterhold associated with the relevant gripper.

6. The device of claim 1, further comprising means for controlled and mutually coordinated movement of the support through the receiving zone under a rotational movement of the receiving wheel during a reception of the objects from the support.

7. The device of claim 1, wherein the supports are guided, in a region of the receiving zone, such that the supports undergo bending.

8. The device of claim 1, wherein the supports have a passage through the receiving zone.

9. The device of claim 8, wherein the passage is a straight-line passage.

10. The device of claim 1, wherein the objects comprise tubular or bottle like objects.

11. The device of claim 1, wherein the objects comprise syringe barrels.

12. The device of claim 6, wherein the movement of the support and the rotational movement of the receiving wheel take place continuously.
